(19) United States
(12) Patent Application Publication
(10) Pub. No.: US 2022/0030834 A1
(43) Pub. Date: Feb. 3, 2022

(54) SYSTEM FOR DETECTING A PATHOLOGY OF A CAT

(71) Applicant: RUBIX S&I, Toulouse (FR)

(72) Inventors: Jean-Christophe Mifsud, Goudourville (FR); Franck Ben Hamouda, Toulouse (FR); Matthieu Ben Hamouda, Plaisir (FR)

(73) Assignee: RUBIX S&I, Toulouse (FR)

(21) Appl. No.: 17/276,461

(22) PCT Filed: Sep. 17, 2019
(86) PCT No.: PCT/EP2019/074874

§ 371 Date: Mar. 15, 2021

(30) Foreign Application Priority Data
Sep. 26, 2018 (FR) 1858800

(51) Int. Cl. A01K 29/00; G01N 33/493; G01N 33/497

(52) U.S. Cl. CPC A01K 29/005; G01N 33/493; A01K 1/0107; G01N 2033/4975; G01N 2800/042; G01N 33/497

(57) ABSTRACT

A system for detecting at least one pathology of a cat, said system being intended to be mounted in the vicinity of a cat litter and comprising at least one gas sensor configured to measure the amount of at least one gas representing a pathology of a cat, at least one alarm component configured to emit an alarm, and at least one electronic unit configured to activate the alarm component if the amount of measured gas is greater than a predetermined threshold.

### TECHNICAL FIELD

The present invention is directed to the field of animal health, in particular that of cats, and relates to a system for detecting a cat pathology.

In a known manner, a cat can develop different diseases, such as diabetes, or be infected by bacteria. In order to detect such pathologies in cats, one known method consists in taking a urine sample from the cat's bladder. The sample is then analyzed in order to detect the presence of bacteria and thus diagnose a pathology. However, results of such an analysis are only obtained after a few days, especially since the sample is cultured so that the bacteria present in the urine can multiply and be detected. Such a detection method is long.

In addition, such a method has uncertain results. Indeed, some diseases may not be detected by urinalysis. In particular, a cat may urinate irregularly. This lack of regularity in the urine may result from a feeding problem.

In another field, it is known to use a litter for a cat living inside an apartment or a house in order to absorb or eliminate the odor of the cat's feces. Such a litter is in the form of sand that is placed in a litter box or toilet house. The litter absorbs odors and therefore has to be regularly renewed in order to maintain its absorption capacity. Generally, people renew the litter when an odor begins to emanate from the litter. However, some people, especially the elderly, may have a less efficient sense of smell, and therefore the frequency of litter renewal is not adequate.

Incidentally, it is known from US 2012 313786 A1, a device that warns the owner of a cat when the cat has used the litter in order to know when to change the litter. Such a device comprises a sensor that detects the presence of the cat. However, such a device does not optimally determine when the litter needs to be changed. It does not enable a cat pathology to be detected.

The purpose of the invention is therefore to overcome at least part of these drawbacks by providing a new system for detecting pathologies for cats.

### SUMMARY

To this end, the invention relates to a system for detecting at least one cat pathology, said system being arranged to be mounted in the vicinity of a cat litter and comprising at least one gas sensor configured to measure the amount of at least one gas representing a cat pathology, at least one warning member configured to issue a warning, and at least one electronic unit configured to activate the warning member if the amount of gas measured is greater than at least one predetermined threshold.

By virtue of the system according to the invention, it is possible to detect a cat pathology based on the analysis of gases emitted by its urine. Such an analysis is fast and does not require a delay in order to obtain a result. Moreover, the system enables an analysis to be performed over a given period of time and not only at a single time instant, as is the case for a urine sample analysis. By virtue of the invention, it is possible to analyze irregularities in the frequency of use of the litter by the cat, for example, problems with food that is too rich in salt (kibble) that affect the proper functioning of the kidney and significantly reduce the amount and regularity of urine. Finally, the gas analysis also makes it possible to indicate to a user when the litter has to be changed, which is advantageous for people with a poor sense of smell.

According to one embodiment, the gas sensor is configured to measure an amount of ketone representative especially of diabetes. Preferably, the amount of ketone is compared to a predetermined ketone threshold.

According to one alternative embodiment, the gas sensor is configured to measure an amount of ammonia and/or sulfide representative of bacterial infection. Preferably, the amount of ammonia and/or sulfide is compared with a predetermined ammonia and/or sulfide threshold. It goes without saying that the same gas sensor could measure different gases successively or simultaneously.

Preferably, the detection system comprises at least one presence sensor in order to measure the amount of gas when the presence of a cat in the vicinity of the litter is detected. The measurement is particularly relevant because it corresponds to the gas emitted by urine. According to one aspect of the invention, the presence sensor is configured to measure the regularity of use of the litter in order to determine kidney pathologies. When the regularity is abnormal, a warning is advantageously issued.

Advantageously, the detection system comprises at least one temperature sensor in order to optimize measurement of the amount of a gas. Indeed, in the case of an MOS type gas sensor, the temperature measured enables the adjustment of the temperature of the heating layer to be optimized in order to accurately determine the gas measured.

Preferably, the gas sensor is of the MOS type. Thus, the same sensor can measure the amount of different gases.

Advantageously, the warning member is configured to issue a visual, audible and/or computer warning. Thus, a user can readily be informed when a warning is activated. Preferably, the warning comprises information related to the pathology detected.

The invention also relates to an assembly of a litter container and a detection system as previously described, in which the detection system is attached to the container, in particular in a central part of the container. Thus, the system according to the invention can be mounted to any type of existing container, which makes it possible to limit costs since only one detection system is required to perform such a detection. A central positioning enables optimal measurement of urine-related gases.

The invention further relates to a method for detecting a cat pathology by means of a detection system as previously described, positioned in the vicinity of a cat litter, said method comprising a step of measuring, in the vicinity of the cat litter, an amount of at least one gas representative of a pathology, a step of comparing the measured amount of gas with at least one predetermined threshold, and a step of issuing a warning in case said threshold is exceeded. Such a method is thus low-time consuming, which makes it possible to issue a warning quickly without waiting.

### BRIEF DESCRIPTION OF THE DRAWINGS

The invention will be better understood upon reading the following description, given only by way of example, and referring to the appended drawings in which:

FIG. 1 schematically represents one embodiment of a detection system according to the invention,

FIG. 2 schematically represents a litter box in which a detection system of FIG. 1 is mounted,

FIG. 3 schematically represents a toilet house in which a detection system of FIG. 1 is mounted, and

FIG. 4 schematically represents one implementation of the detection method according to the invention.

It should be noted that the figures set forth the invention in a detailed manner to implement the invention, said figures can of course be used to better define the invention if necessary.

### DETAILED DESCRIPTION

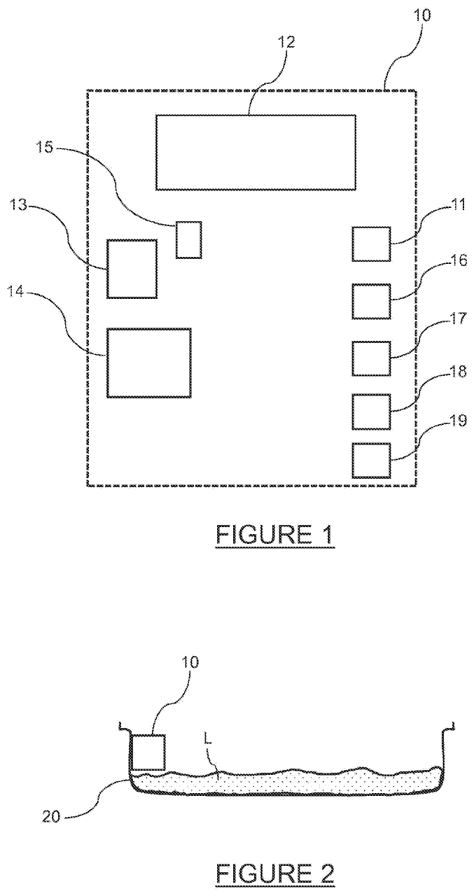

With reference to FIG. 1, a system 10 for detecting at least one cat pathology, adapted to detect and measure the amount of gas in the air in the vicinity of a cat litter, is schematically represented. Such gases are representative of different cat pathologies, such as diabetes, the presence of bacteria, poor nutrition, etc.

In this embodiment, the detection system 10 comprises a gas sensor 11, a warning member 12 and an electronic unit 13 controlling the warning member 12.

The gas sensor 11 is configured to detect a gas present in the air in the vicinity of the litter and to measure the amount thereof. The detected gas comes from the cat's urine, which enables the cat to be diagnosed. The gas sensor 11 can be configured to detect gas on an ad hoc or periodic basis as described in the following to monitor the cat.

The gas sensor 11 detects at least one gas representative of a pathology in the cat. Such a gas can especially be:
ammonia or aliphatic amines that are characteristic of a pathology related to bacterial contamination,
branched chain fatty acids, such as butyric acid or 2-amino-7-hydroxy-5,5-dimethyl-4-thiaheptanoic acid, also known as felinine, which are characteristic of a hormonal variation especially after castration in male cats,
phenols, such as ethylphenol, para cresol, ethanol or n-propanol, and/or
volatile sulfur compounds, such as dimethyl sulfide, dimethyl disulfide, 3-mercapto-3-methyl-1-butanol, which are characteristic of an abnormal variation in hormone levels.

When the amount of such gases exceeds one or more predetermined thresholds associated with said measured gases, they are representative of a pathology in cats, especially diabetes, bacterial infection, etc. Preferably, the gas sensor 11 is configured to perform multi-gas measurements and comparisons having several predetermined thresholds to determine a signature representative of a disease.

In one embodiment according to the invention, the gas sensor is an oxido-metallic sensor, also referred to as MOS. Such an MOS sensor comprises a layer sensitive to at least one gas and a heating layer mounted to said sensitive layer.

The sensitive layer is in contact with the air in the vicinity of the litter and is adapted to absorb molecules of the gas to be measured. The absorption of these molecules varies the electrical conductivity through the sensitive layer by virtue of an oxidation-reduction reaction, which makes it possible to determine the amount of said gas present in the air in contact with the sensitive layer. In this case, the amount of gas is measured from the variation in the resistance dR with respect to the initial resistance Rₒ of the MOS sensor. In other words, the amount of gas measured is equal to the ratio dR/Rₒ.

The heating layer is controlled, preferably by the electronic unit 13, in order to modify the temperature of the sensitive layer, which enables the sensitive layer to absorb different gases: each temperature of the sensitive layer enabling the amount of a gas to be measured. By virtue of the heating layer, only one sensitive layer enables different gases to be measured, which limits electric energy consumption and cost of the MOS sensor.

The detection system 10 could also comprise more than one MOS sensor. For example, MOS sensors with a different oxide in order to detect different elements of the gas to be measured and thus optimize measurements and increase the relevance of the detection. An MOS type sensor has been set forth but of course an electrochemical sensor could also be suitable, especially an electrochemical sensor sensitive to different gases such as ammonia.

The warning member 12 is configured to issue a warning when one or more predetermined thresholds are exceeded. In other words, a pathology is associated with a combination of threshold exceeding events.

Such a warning can be audible or visual and the warning member 12 can comprise an audible alarm, at least one diode and/or a display screen. Thus, a user is informed when a pathology is detected. Preferably, the warning member 12 is configured to issue a warning corresponding to the detected pathology. For example, diodes of different colors can be provided depending on the detected pathology.

According to one embodiment, the warning member 12 can also comprise a preferably wireless, especially Bluetooth communication module. The communication module is then configured to communicate with a user's terminal, such as his/her smartphone, so that the user receives warnings thereon.

The warning member 12 can also display the pathology detected, especially on the display screen of the warning member or on the terminal.

The electronic unit 13 controls the warning member 12 on the basis of measurements performed by the gas sensor 11.

For this purpose, the electronic unit 13 is configured to receive measurements performed by the gas sensor 11, to compare these measurements with predetermined thresholds and to control the warning member 12 to issue a warning when a measurement exceeds one or more predetermined thresholds.

The electronic unit 13 can also control the MOS type gas sensor 11, especially to control the gas to be measured. For this purpose, the electronic unit 13 is configured to control the temperature of the heating layer of the MOS sensor. Thus, the gas sensor 11 measures the gas(es) representative of the desired pathology. Preferably, the electronic unit is in the form of a microprocessor.

According to one embodiment, the detection system 10 further comprises an energy storage unit 14, such as a battery or a cell, in order to supply the various elements of the detection system with electric energy, in particular the gas sensor 11, the warning member 12 or the electronic unit 13, and thus ensure autonomy of the detection system 10 in terms of electric energy.

The detection system 10 can also comprise a storage unit 15, such as a memory, to record the measurements performed. This makes it possible to monitor the measurements, especially to analyze the variation in the amount of different gases over time. Thus, a pathology can be detected at an early stage. The predetermined thresholds can also be recorded in the storage unit 15.

The detection system 10 can comprise other sensors in order to optimize the detection of a pathology. In particular, the detection system 10 can comprise a temperature sensor 16 configured to measure the temperature in the vicinity of the litter. Such a temperature sensor 16 also enables the temperature of the heating layer of the MOS sensor to be adjusted according to the temperature measured by the temperature sensor 16.

A humidity sensor 17 can also be used to detect a cat's urine. This makes it possible to detect the absence of urine for a period of time, which can be representative of a pathology. In addition, the gas measurement can be activated when urine has been detected. Thus, measurements are performed at the optimal time, that is as soon as urine is present. This limits the risk of gas not being detected, especially when it is absorbed by the litter or evaporates. Furthermore, it limits the electricity consumption by only carrying out relevant measurements.

A presence sensor 18 can be configured to trigger a gas measurement when the cat arrives in the vicinity of the litter. Thus, measurements are performed at the optimal time, that is when the cat is urinating. Indeed, since the litter absorbs urine, which leads to the absorption or destruction of gases, gas measurement has to be carried out before such absorption or destruction. Moreover, presence detection avoids false gas detections due to a persistent odor and not to the cat's urine.

Finally, the detection system 10 can comprise a particle sensor 19 configured to detect particles in the air. Such particles are especially silicate alumina and/or silica particles that may be released when handling the litter. Since these particles can cause cancer, especially of the sarcoidosis type, the warning member 12 is configured to issue a warning regarding the risks associated with these particles when they are detected. Analogously, particle measurements are compared with one or more predetermined thresholds.

The detection system 10 is configured to be mounted to a litter container in order to be in proximity to the litter used by a cat and thus detect a pathology in the cat's urine fumes. The detection system 10 adapts to any type of container so that it can be retrofitted to an existing container. The detection system 10 is detachably mounted in order to be subsequently mounted to another container, for example, by means of a clip or similar.

With reference to FIG. 2 a container in the form of a litter box 20 is schematically represented, in which a litter L has been put. Such a litter box 20 comprises a wall defining a cavity in which the litter L is placed. The detection system 10 can then be mounted to the wall so as not to inconvenience the cat while remaining in the vicinity of the urine fumes emanating from the litter L.

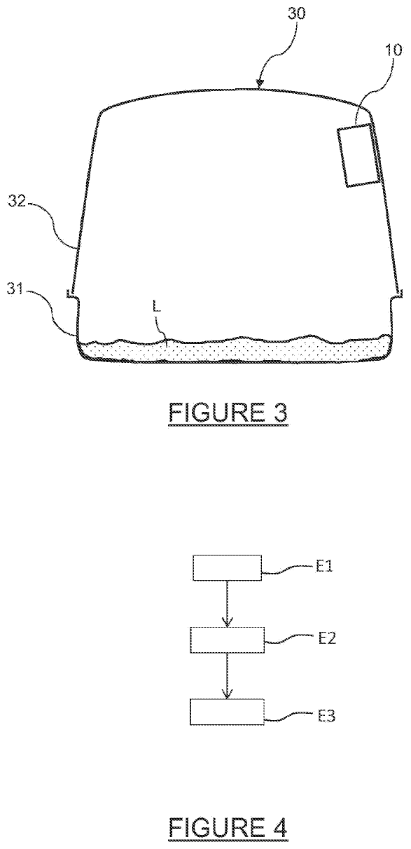

With reference to FIG. 3, a container in the form of a toilet house 30 is schematically represented. Such a toilet house 30 comprises a lower part 31 and an upper part 32. The lower part 31 has a shape similar to a litter box in which the litter L is placed. The upper part 32 is mounted to the lower part 31 to form a roof covering the litter L. Thus, the litter is placed in an enclosure in which urine fumes are diffused. The detection system 10 is advantageously mounted to the upper part 32, in particular inside the same, in order to optimize gas detection in the enclosure of the toilet house 30. Thus detection is optimal and independent of the zone in which the cat has urinated.

The toilet house 30 can also comprise an access cat flap through which the cat enters the toilet house to urinate. The presence sensor 18 of the detection system 10 can then be configured to detect opening of the access cat flap and thus control a relevant measurement of the amount of gas subsequently to opening the access cat flap.

With reference to FIG. 4, an example of implementation of the method according to the invention for detecting a pathology using a detection system 10 will now be described.

To measure gases, the user mounts the detection system 10 in the vicinity of the litter used by the cat in whom he/she wishes to detect a pathology. For this purpose, the detection system 10 is mounted in a litter box 20 or even in an existing toilet house 30.

When using the detection system 10, the presence sensor 18 detects the presence of a cat at the litter L. The gas sensor 11 then measures, in a step E1, the amount of different gases, such as ammonia, sulfide and/or ketone, in air.

The electronic unit 13 then receives the measurements and compares them to predetermined thresholds in a step E2. If the amount of a gas is higher than its associated threshold, the electronic unit 13 activates, in a step E3, the warning member 12. The warning member 12 then issues an audible, visual or computer warning via the wireless communication module. The user is thus informed that a pathology has been detected.

The measurement of the amount of a gas by the gas sensor 11 following the detection of a presence has been described. However, such a measurement could of course be carried out on an ad hoc basis or at regular time intervals in order to perform a continuous detection over a period of time. A continuous detection also enables a drop in the frequency of the cat's urine to be detected, which is a sign of a nutrition disorder, especially an excessively salty diet.

According to one aspect of the invention, the electronic unit 13 records measurements performed by the various sensors 11, 16, 17, 18, 19. The electronic unit 13 can thus activate the warning member 12 if it has been detected, from the presence sensor 18 and/or the gas sensor 11, that the cat has not urinated for a long period of time. Likewise, the electronic unit 13 can activate the warning member 12 if it has detected, from the gas sensor 11, that the litter has to be changed.

1-9. (canceled)

10. A system for detecting at least one cat pathology, said system being arranged to be mounted in the vicinity of a cat litter and comprising:
at least one gas sensor configured to measure the amount of at least one gas representative of a cat pathology,
at least one warning member configured to issue a warning, and
at least one electronic unit configured to activate the warning member if the amount of gas measured is greater than at least one predetermined threshold.

11. The detection system according to claim 10, wherein the gas sensor is configured to measure an amount of ketone representative of diabetes.

12. The detection system according to claim 10, wherein the gas sensor is configured to measure an amount of ammonia and/or sulfide representative of bacterial infection.

13. The detection system according to claim 10, comprising at least one presence sensor.

14. The detection system according to claim 10, comprising at least one temperature sensor.

15. The detection system according to claim 10, wherein the gas sensor is of the MOS type.

16. The detection system according to claim 10, wherein the warning member is configured to issue a visual, audible and/or computer warning.

17. An assembly of a litter container and the detection system according to claim 10, wherein the detection system is attached to the container, in particular in a central part of the container.

18. A method for detecting a cat pathology by means of the detection system according to claim 10 positioned in the vicinity of a cat litter, said method comprising:
a step of measuring, in the vicinity of the cat litter, an amount of at least one gas representative of a pathology,
a step of comparing the amount of gas measured with at least one predetermined threshold, and
a step of issuing a warning in case of said threshold being exceeded.
